(19) United States
(12) Patent Application Publication
(10) Pub. No.: US 2021/0007750 A1
(43) Pub. Date: Jan. 14, 2021

(54) Coaxial Coil Detachment Systems Based on Targeting and Shock Absorbing Coil Improvements

(71) Applicant: BALT USA, Irvine, CA (US)

(72) Inventors: Jake Le, Foothill Ranch, CA (US); Stephanie Gong, Aliso Viejo, CA (US)

(21) Appl. No.: 16/927,832

(22) Filed: Jul. 13, 2020

Related U.S. Application Data
(60) Provisional application No. 62/873,893, filed on Jul. 13, 2019.

(51) Int. Cl. A61B 17/12

(52) U.S. Cl. CPC A61B 17/1214; A61B 2017/00526; A61B 2017/12068

(57) ABSTRACT

Improved thermal detachment driven by leverage from improved electrical locus targeting enabling first button detachment at optimal regions whereby, for example OPTIMA® brands of coils (BALT USA®, Orange County, CA 92618) and their detachment systems are shifted and realigned coaxially and placed closer to the ends of subject coils while being hermetically sealed,

### CROSS REFERENCE TO RELATED APPLICATIONS

The application claims full priority benefit from U.S. Ser. No. 62/873,893, filed Jul. 13, 2019. Likewise, expressly incorporated by reference are the following: Ser. No. 15/537,881, 15/372,881, 15/400,897,15/106,262, 15/771,187, 16/476,524, 62/975,163, 16/862,449, 63/035,075, and 9,566,071 and 9,566,072.

### BACKGROUND OF THE DISCLOSURES

Detachment of, for example, coils and related systems within the cerebral neurovascular space is fraught with issues and challenges. The present inventions overcome broken lead wires and are now in patients and the market, delivering first button actuated detachment.

### OBJECTS AND SUMMARY OF THE INVENTION

Briefly stated, improved shock absorbers in thermal detachment driven by leverage from improved electrical locus targeting and related applications of the same allows electron density to define Optimal Regions balancing the resistance and heat whereby, for example OPTIMA® brands of coils (BALT USA®, Orange County, Calif.) and their detachment systems are from shifted coaxially and placed closer to the ends of subject coils.

According to embodiments, there is disclosed a novel enhanced system with improved spacing for shock absorption for leveraging the most electron dense locus in a thermal detachment system, which comprises, in combination, mapping the precise zone, or locus where a heater coil becomes most heated, frame-shifting said locus to a proximal portion of said coil, severing a requisite SR thread, closer to the front than conventional systems, and reducing or eliminating any dragger or tail region based upon said frame shifting.

According to embodiments, there is disclosed a system, further comprising, “Coaxialization” (as defined herein) of the frame shifted locus within said coil.

According to embodiments, there is disclosed a system of the instant disclosure in total, wherein, any mushroom or the-like shaped artifacts within subject marker bands are mitigated, reduced or eliminated, by the improved processing disclosed herein.

According to embodiments, there is disclosed the system of the figures, appendices and the instant disclosure in total further comprising, hermetically sealed coils.

According to embodiments, there is disclosed the system of claims 1-4 and the instant disclosure in total, whereby, the solution of coaxializing the detachment end of said coil renders the hottest point at the tip by distal movement and folding.

### BRIEF DESCRIPTION OF THE DRAWINGS

Various preferred embodiments are described herein with references to the drawings in which merely illustrative views are offered for consideration, whereby:

FIG. 1 shows the prior art design improved upon by the present inventions;

FIG. 2 shows a root cause of prior art non-detachment wherein higher tensile force loaded onto one lead wire caused failures;

FIG. 3 shows where prior art failure occurs via broken lead wire;

FIG. 4 shows gaps to allow for chock absorption, distributing force on both lead wires;

FIG. 5 illustrates results of the coaxial shock absorbing coil of the present invention;

FIG. 6 shows the improved heater coil, along with manufacturing steps of middle pinch, making coaxial and location of heat locus at distal end;

FIG. 7 shows why first attempt detachment happens with the new coaxial heater coil design, which heats at distal end; and

FIG. 8 shows a new coaxial design heated at distal end resulting in very reliable detachment on first attempt.

Corresponding reference characters indicate corresponding components throughout the several views of the drawings. Skilled artisans will appreciate that elements in the figures are illustrated for simplicity and clarity, and have not necessarily been drawn to scale. For example, the dimensions of some of the elements in the figures may be exaggerated relative to other elements to help to improve understanding of various embodiments of the present invention.

Also, common but well-understood elements that are useful or necessary in a commercially feasible embodiment are often not depicted in order to facilitate a less obstructed view of these various embodiments of the present invention.

### DETAILED DESCRIPTIONS

The present inventors have realized that using a shock absorbing mechanical lead wire failure is called for and the problems of the prior art cured for the arrangement work together with optimized mapping of electron density which thus allows them to realize more efficient and reliable detachment among, for example thermal detachments systems such as those available (OPTIMA®, BALT USA, Orange County, Calif. 92618, to treat aneurysms et seq. Turning now to the figures.

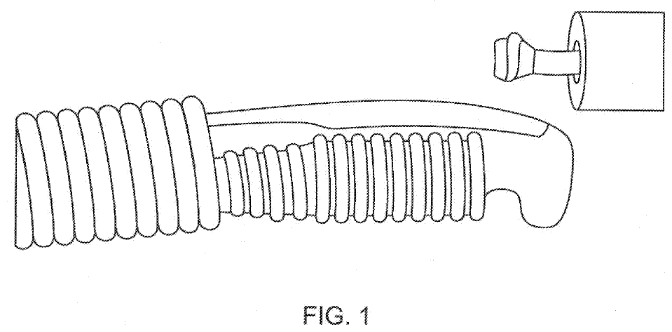

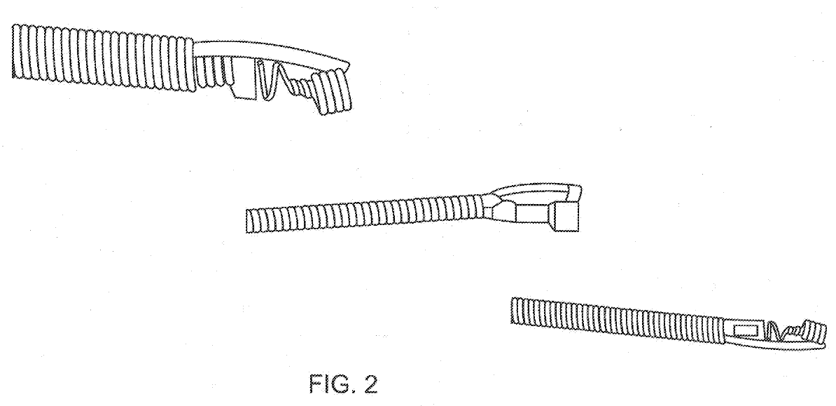

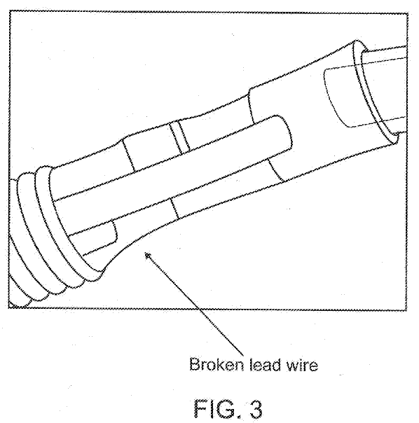

FIG. 1-5 each shows the current design and prior art which motivated the change. During a sustaining engineering review of non-detachment (FIG. 2 and FIG. 3) it was discovered that lead wires were detaching.

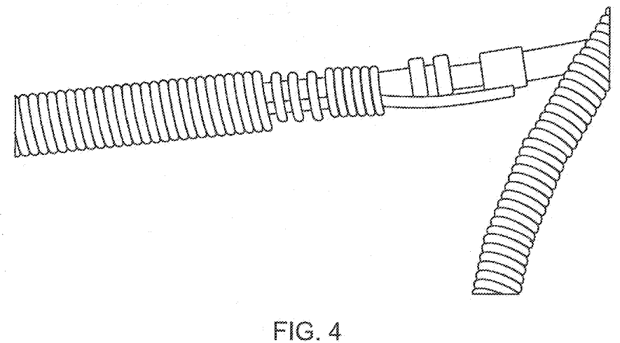

To eliminate this issue FIG. 4 shows that SHOCK ABSORBING COILS (i) distribute tensile forces on both lead wires, (ii) lead to first time detachment figures also.

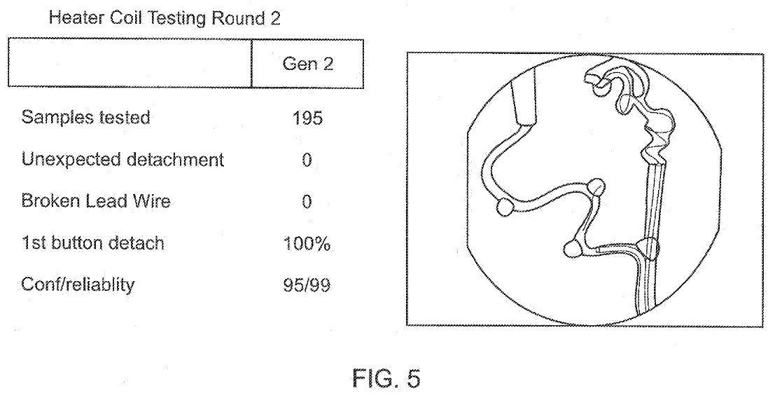

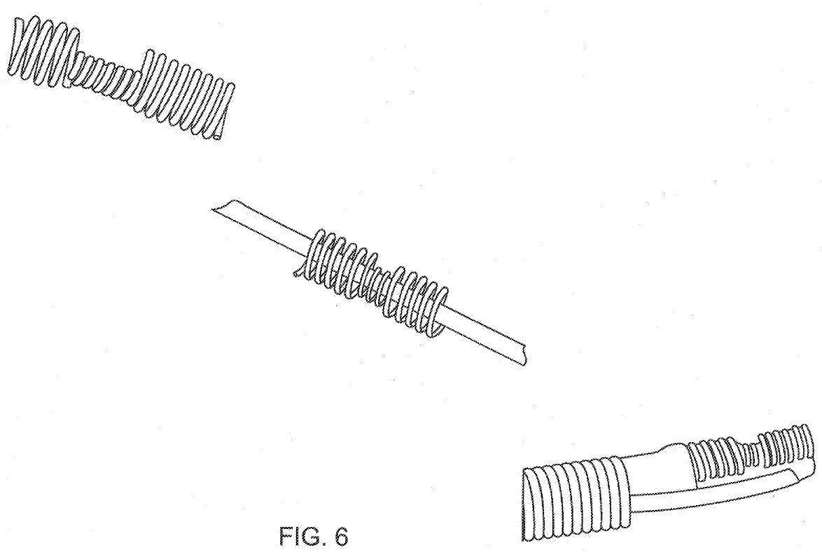

FIG. 5-7 show the results of Heater Coil Testing, round second where the issue was eliminated. Thus the present invention solves the problem and is now part of the system.

Referring now to FIG. 1. Heater coils melt (SR) threads—this allows implant to separate from pusher.

The heater coil is designed to melt the stretch resistance (SR) thread. This allows the implant to separate from the pusher.

Electrical current is delivered from BALT USA's XCEL Controller®;

The heater coil is designed to be resistive to current, which causes the heater coil get hot.

The heater coil is designed to heat up at the center of the coil.

The heater generated by the heater coil melts the stretch resistance thread, which allows the implant to be detached from the pusher.

Referring to FIG. 5 and all other figures:

The middle of the heater coil is pinched to a smaller diameter;

The distal end of the heater coil is pushed over the pinched section;

A first layer of insulation jacket is shrunk over the heater coil.

Referring to FIG. 5 and all other figures:

A 2^{nd} layer of insulation jacket is shrunk over the heater coil;

Several gaps are created proximal to the heater coil;

The result allows the heater coil to get hot at the distal end of the pusher.

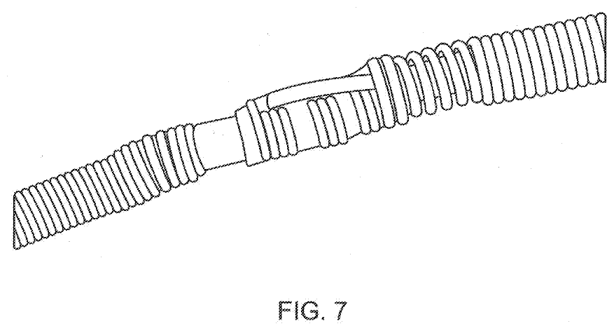

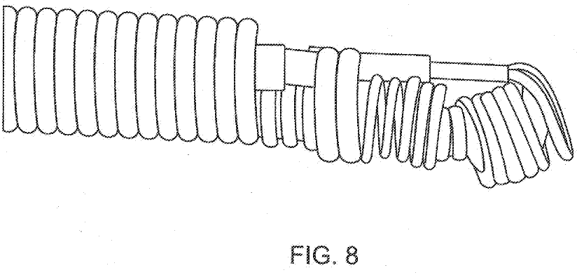

Referring now to FIG. 7 and FIG. 8 first button detachability is improved because, as shown with FIG. 7, the new coaxial heater coil the implant (bulbous) detaches on first attempt.

FIG. 8 shows the coaxial design heating at distal end and reliably separating.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Reference throughout this specification to “one embodiment,” “an embodiment,” or similar language mans that a particular feature, structure, or characteristic described in connection with the embodiment is included in at least one embodiment of the present invention. Thus, appearances of the phrases “in one embodiment,” “in an embodiment,” and similar throughout this specification may, but do not necessarily, all refer to the same embodiment.

Furthermore, the described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention may be practiced without one or more of the specific details, or with other methods, components, materials, and so forth. In other instances, well-known structures, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

A processor may be provided by one or more processors including, for example, one or more of a single core or multi-core processor (e.g., AMD Phenom II X2, Intel Core Duo, AMD Phenom II X4, Intel Core i5, Intel Core I & Extreme Edition 980X, or Intel Xeon E7-2820).

An I/O mechanism may include a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), an accelerometer, a microphone, a cellular radio frequency antenna, and a network interface device (e.g., a network interface card (NIC), Wi-Fi card, cellular modem, data jack, Ethernet port, modem jack, HDMI port, mini-HDMI port, USB port), touchscreen (e.g., CRT, LCD, LED, AMOLED, Super AMOLED), pointing device, trackpad, light (e.g., LED), light/image projection device, or a combination thereof.

Memory according to the invention refers to a non-transitory memory which is provided by one or more tangible devices which preferably include one or more machine-readable medium on which is stored one or more sets of instructions (e.g., software) embodying any one or more of the methodologies or functions described herein. The software may also reside, completely or at least partially, within the main memory, processor, or both during execution thereof by a computer within system, the main memory and the processor also constituting machine-readable media. The software may further be transmitted or received over a network via the network interface device.

While the machine-readable medium can in an exemplary embodiment be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present invention. Memory may be, for example, one or more of a hard disk drive, solid state drive (SSD), an optical disc, flash memory, zip disk, tape drive, “cloud” storage location, or a combination thereof. In certain embodiments, a device of the invention includes a tangible, non-transitory computer readable medium for memory. Exemplary devices for use as memory include semiconductor memory devices, (e.g., EPROM, EEPROM, solid state drive (SSD), and flash memory devices e.g., SD, micro SD. SDXC. SDIO, SDHC cards); magnetic disks, (e.g., internal hard disks or removable disks); and optical disks (e.g., CD and DVD disks).

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A coaxial coil detachment system, comprising, in combination:
a heater coil which gets hot at a distal end owing to a configuration of a new pinched section of the heater coil.

2. The coaxial coil detachment system of claim 1, whereby reliable detachment occurs on the first attempt.

3. The coaxial coil detachment system of claim 2, whereby the coil is imposed by pinching in the middle which eliminates non-detachment from broken lead wires.

4. The coaxial coil detachment system of claim 3, whereby the distal end of the heater coil is pushed over the pinched section.

5. The coaxial coil detachment system of claim 4, where a first layer of insulation jacket is shrunk over the heater coil.

6. The coaxial coil detachment system of claim 5, wherein a 2^{nd} layer of insulation jacket is shrunk over the heater coil.

7. The coaxial coil detachment system of claim 6, wherein several gaps are created proximal to the heater coil.

8. The coaxial coil detachment system of claim 7, whereby the resultory configuration forces the heater coil to get hot at the distal end of the pusher.

9. The coaxial coil detachment system of claim 8, with no unexpected detachment from broken lead wires.

10. The coaxial coil detachment system of claim 9, with 100% reliability of first button detachment attempts completed.

11. A method of manufacturing a new coaxial heater and shock absorbing coil, which distributes force onto both lead wires, comprising, in combination:
pinching a middle section of a heater coil to a smaller diameter;
pushing a distal end of the heater coil over the inched section;
shrinking a first layer of insulation jacket over the heater coil;
shrinking a second layer of insulation over the heater coil.

12. The method of manufacturing of claim 11, wherein several gaps are created proximal to the heater coil.

13. The method of manufacturing of claim 12, resultory heater coils getting hotter at the distal end of a pusher.

14. Products, by the method of claim 13, wherein first button detachment reliability is achieved namely implants separate from pusher on first detachment attempt.

15. A process for first attempt detachment compliance, which comprises, in combination:
providing a heater coil which does not get hot at the center, rather at a distal and from the new geometry applied-namely configuration of a pinched section of the heater coil.

16. The process of claim 15, further comprising:
the providing step supplies an Optima® brand of system from BALT USA (Orange County, Calif. 92618).
